(19) United States
(12) Patent Application Publication
(10) Pub. No.: US 2008/0188003 A1
(43) Pub. Date: Aug. 7, 2008

(54) Vinyl ends method

(76) Inventor: Arthur Thaler Spaugh, Kingsport, TN (US)

Correspondence Address: STEVEN A. OWEN;EASTMAN CHEMICAL COMPANY, P.O. BOX 511, KINGSPORT, TN, 37662, US

(21) Appl. No.: 11/701,599

(22) Filed: Feb. 2, 2007

(51) Int. Cl. G01N 33/44

(52) U.S. Cl. 436/85

(57) ABSTRACT

A method for the measurement of vinyl ends concentration in a polyester polymer by dissolving the polymer in a liquid solution with fluorinated carboxylic acid or fluorinated carboxylic anhydride, optionally heating the liquid solution or the mixture to a temperature between about 30° C. and 300° C., subjecting the resulting mixture to an analysis where a quantitative signal for a fluorine atom or a fluorine-containing compound is produced, and calculating the quantity or concentration of vinyl ends from said quantitative signals.

### 1. FIELD OF THE INVENTION

The invention relates to a method for the measurement of vinyl ends concentration in a polyester polymer. More particularly, the invention relates to a method for the measurement of vinyl ends in a polyester polymer using a liquid solution comprising at least one fluorinated carboxylic acid or fluorinated carboxylic anhydride to form a mixture, optionally heating the liquid solution or the mixture to a temperature between about 30° C. and 300° C., subjecting said mixture to an analysis wherein a quantitative signal for a fluorine atom or a fluorine-containing compound is produced; and calculating the quantity or concentration of vinyl ends from said quantitative signals.

### 2. BACKGROUND OF THE INVENTION

Polyester polymers and especially polyethylene terephthalate polymer are widely used for various applications such as sheets, boards, extrusion blow molded bottles, extruded laminates, containers, and beverage bottles. Certain physical characteristics make polyester polymers and polyester polymer particles such as polyethylene terephthalate (PET) desirable for packaging applications include impact strength, moldability, clarity, transparency, and color. However, depending upon the specific application, there are other characteristics and properties that are desirable especially for extrusion blow molded articles.

For example, polyester polymers and especially PET (polyethylene terephthalate) is used extensively in water bottle and carbonated soft drink (“CSD”) bottle applications. A typical method by which these bottles are produced is by melt processing of PET pellets to form bottle preforms followed by extrusion blow molding to form bottles. In this application, concentration levels of acetaldehyde (AA) are a concern. Of specific concern are two types of acetaldehyde (AA). The first is residual or free AA contained in polyester pellets or polyester particles used as raw material in extrusion blow molding. A second type of AA is preform AA or the AA generated when PET pellets are melt processed to make bottle preforms. AA precursors in the solid polyester particles, chemical compounds or chemical functional groups which may react upon degradation and/or upon melting of the polyester can produce unacceptable levels of AA in the preforms. In addition, new AA precursors are formed when the polyester polymer is held in the molten state, as in the case of an injection molding process to make bottle preforms. Acetaldehyde has a noticeable taste and can be highly undesirable in beverage container applications. When performs are blown into bottles, unacceptably high AA levels are those that adversely impact the taste of the beverage contained in these bottles. Relatively tasteless beverages such as water are particularly negatively impacted by the taste of AA. Many water bottle applications require lower levels of preform AA than carbonated soft drink (“CSD”) bottle applications.

Another example of a normally desirable characteristic of the polyester polymer melts and any subsequent polyester particles produced by solidification of the melt is that of low vinyl ends concentration. Vinyl ends as represented by the formula: —CO₂—CH=CH₂ are known AA precursors. One commonly accepted mechanism by which AA is generated in molten polyester is the internal chain scission of a polyester polymer chain to form a vinyl end group and a carboxylic acid end group. The vinyl end group can react with a hydroxyethyl end group or water to form residual or free AA and a new internal ester linkage. There is a common perception that a high concentration of vinyl ends is thus undesirable due to the ability of the vinyl end to react to form AA during subsequent melt processing of the polyester polymer.

Because subsequent production of AA can be problematic, there are many advantages in having an accurate quantitative measurement of the concentration of AA precursors, and specifically a quantitative measurement of the concentration of vinyl ends. For example, by having a quantitative measurement of vinyl ends, it may be advantageous for bottle producers to adjust the operating conditions of extrusion blow molding equipment (e.g. operating temperature, residence time inter alia) to minimize the amount of AA generated during the molding process. In another example, by having a quantitative measurement of vinyl ends, it may be advantageous for PET pellet producers to adjust the operating conditions of known solid state polymerization processes to remove sufficient AA to make the polyester polymer product acceptable for water bottle and/or CSD applications. In still another example, by having a quantitative measurement of vinyl ends, it may be advantageous to adjust the amount of AA scavenger added to a polyester polymer to match the amount of AA generated anticipated from decomposition of vinyl ends.

Other than AA generation, there are other advantages of having a quantitative measurement of vinyl ends concentration in polyester polymers. For example, a smaller number of olefin terminals (e.g. vinyl ends) is usually preferred for improving melt heat stability of the modified and unmodified polyester polymers. In particular, it is usually undesirable to have large variations in intrinsic viscosity between pellets, preforms, and/or bottles since this could lead to inconsistency in the end application. For beverage applications such as carbonated soft drink or water bottles there is usually is not more than 0.04 dL/g, preferably not more than 0.03 dL/g, and most preferably not more than 0.02 dL/g difference in intrinsic viscosity. Additionally, it is known that vinyl ends may also polymerize to polyvinyl esters which may be responsible for yellow coloration of PET.

There are known methods for measuring olefin terminal ends concentration in highly modified polyester polymers are reported such as reported in J. Polymer Science A, volume 39, issue 5, 665-674 (reference A). However, the methods described are generally associated with the measurement of cyclovinylidene and methyl cyclohexene end groups rather than vinyl ends. The cyclovinylidene and methyl cyclohexene end groups are characteristic of cyclohexanedimethanol-modified polyethylene terephthalate polymers. Reference A does refer to two methods of measuring vinyl ends concentration. Both methods described have significant shortcomings. The first method utilizes taking a proton 1H-NMR (proton nuclear magnetic resonance) spectra. The second method is a modified method based on a journal article (J. G. M. Aalbers, and G. D. B. van Houwelingen, Fresenius Z Anal. Chem., 314(5), 472-475(1983)) wherein vinyl ends are reacted with bromine and followed by measurement of coulometric bromination. This method requires a second measurement to correct for other compounds which react with bromine. The method is not specific for the analysis of the vinyl end group and even with the correction described in the method; the results will be confounded by other reactions which consume bromine.

As reported in Reference A, the amount of vinyl ends in PET homopolymer is small compared with the content of cyclovinylidene and methyl cyclohexene end groups in cyclohexanedimethanol-modified polymers. Further Reference A also indicate that quantitative differences in the values of vinyl ends concentration using both methods for PET homopolymer may be either a result of experimental error or a result of the presence of other unsaturated species other than vinyl ends.

These methods are not entirely satisfactory because they have poor sensititivity toward detecting vinyl ends at low concentration levels and one method is not specific toward the detection of vinyl ends only. Hence, there is a need to develop a method for measurement of vinyl ends concentration in polyester polymers and particularly polyethylene terephthalate polymer that is specific toward measurement of vinyl ends, that is sensitive and accurate to low concentration levels of vinyl ends, and that can be effectively utilized when measuring vinyl ends in PET homopolymers.

### 3. SUMMARY OF THE INVENTION

An objective of this invention is to provide a method for the measurement of vinyl ends concentration in a polyester polymer. In an embodiment of this invention, a method for the measurement of vinyl ends concentration in a polyester polymer is provided comprising:
a). dissolving said polyester polymer with a liquid solution comprising at least one fluorinated carboxylic acid or fluorinated carboxylic anhydride to form an anhydrous mixture;
b). subjecting said mixture to an analysis wherein a quantitative signal for fluorine or a fluorine-containing compound is produced; and
c). calculating the quantity or concentration of vinyl ends from said quantitative signals.

In another embodiment of this invention, a method for the measurement of vinyl ends concentration in a polyester polymer is provided comprising:
a). dissolving said polyester polymer with a liquid solution comprising at least one fluorinated carboxylic acid or fluorinated carboxylic anhydride to form an anhydrous mixture;
b). optionally heating said liquid solution or said mixture to a temperature between about 30° C. and 300° C.;
c). subjecting said mixture to an analysis wherein a quantitative signal for fluorine or a fluorine-containing compound is produced; and
d). calculating the quantity or concentration of vinyl ends from said quantitative signals.

### 4. BRIEF DESCRIPTION OF THE DRAWINGS

FIG. 1 is an NMR spectra showing a vinyl ends concentration of Example 1.

FIG. 2 is an expansion of the NMR spectra of FIG. 1.

### 5. DETAILED DESCRIPTION OF THE INVENTION

The present invention may be understood more readily by reference to the following detailed description of the invention.

In the method of this invention, a “polyester polymer” comprises a dicarboxylic acid moiety comprising one or more dicarboxylic acids or their ester forming derivatives and a diol moiety. Examples of dicarboxylic acid moieties include, but are not limited to, terephthalic acid, derivates of terephthalic acid, isophthalic acid, derivates of isophthalic acid, naphthalene-2,6-dicarboxylic acid, derivatives of naphthalene-2,6-dicarboxylic acid, or mixtures thereof. Other examples of dicarboxylic acid moieties include, aromatic dicarboxylic acids having 8 to 14 carbon atoms, aliphatic dicarboxylic acids having 4 to 12 carbon atoms, or cycloaliphatic dicarboxylic acids having 8 to 12 carbon atoms.

Examples of diol moieties include, but are not limited to, ethylene glycol, cycloaliphatic diols preferably having 6 to 20 carbon atoms and/or aliphatic diols preferably having 3 to 20 carbon atoms. More specific examples of such diols include diethylene glycol; triethylene glycol; 1,4-cyclohexanedimethanol; propane-1,3-diol; butane-1,4-diol; pentane-1,5-diol; hexane-1,6-diol; 3-methylpentanediol-(2,4); 2-methylpentanediol-(1,4); 2,2,4-trimethylpentane-diol-(1,3); 2,5-ethylhexanediol-(1,3); 2,2-diethyl propane-diol-(1,3); hexanediol-(1,3); 1,4-di-(hydroxyethoxy)-benzene; 2,2-bis-(4-hydroxycyclohexyl)-propane; 2,4-dihydroxy-1,1,3,3-tetramethyl-cyclobutane; 2,2-bis-(3-hydroxyethoxyphenyl)-propane; and 2,2-bis-(4-hydroxypropoxyphenyl)-propane, and diethylene glycol.

Of particular interest are polyester polymers containing repeating alkylene aryl units, such as alkylene terephthalate or alkylene naphthalate repeat units in the polymer chain. More specific examples of these repeating units include ethylene terephthalate, ethylene naphthalate, and trimethylene terephthalate. An example of a polyester polymer is one which comprises:
(i) a carboxylic acid component comprising at least 80 mole % of the residues of terephthalic acid, derivates of terephthalic acid, naphthalene-2,6-dicarboxylic acid, derivatives of naphthalene-2,6-dicarboxylic acid, or mixtures thereof, and
(ii) a hydroxyl component comprising at least 80 mole % of the residues of ethylene glycol or propane diol,
   based on 100 mole percent of carboxylic acid component residues and 100 mole percent of hydroxyl component residues in the polyester polymer.

All compounds containing carboxylic acid group(s) or derivative(s) thereof that are part of the polyester polymer comprise the “carboxylic acid component residue.” The mole % of all the compounds containing carboxylic acid group(s) or derivative(s) thereof which are in the polyester polymer sum to 100.

All compounds containing hydroxyl group(s) or derivatives thereof that are part of the polyester polymer comprise the hydroxyl component. The mole % of all the compounds containing hydroxyl group(s) or derivatives thereof which are in the polyester polymer sum to 100.

Another example of a polyester polymer is one which comprises:
(a) a carboxylic acid component comprising at least 90 mole %, or at least 92 mole %, or at least 96 mole % of the residues of terephthalic acid, derivates of terephthalic acid, naphthalene-2,6-dicarboxylic acid, derivatives of naphthalene-2,6-dicarboxylic acid, or mixtures thereof, more preferably terephthalic acid or derivates of terephthalic acid, and
(b) a hydroxyl component comprising at least 90 mole %, or at least 92 mole %, or at least 96 mole % of the residues of ethylene glycol or propane diol, more preferably ethylene glycol, based on 100 mole percent of the carboxylic acid component residues and 100 mole percent of the hydroxyl component residues in the polyester polymer.

In an embodiment of this invention, a method for the measurement of vinyl ends concentration in a polyester polymer is provided.

A polyester polymer is combined with a liquid solution comprising at least one fluorinated carboxylic acid or fluorinated carboxylic anhydride, preferably at least one perfluorinated carboxylic acid or perfluorinated carboxylic anhydride. Fluorinated carboxylic acids generally constitute linear or branched aliphatic or aromatic carboxylic acids, wherein at least one of the carbon-bonded hydrogen atoms has been replaced by fluorine. As it is used herein, the term perfluorinated means that all of the carbon-bonded hydrogen atoms have been replaced by fluorine. Examples of suitable fluorinated acids and anhydrides include, but are not limited to, monofluoroacetic acid, difluoroacetic acid, trifluoroacetic acid, pentafluoropropionic acid, heptafluorobutyric acid, perfluoropentanoic acid, perfluorohexanoic acid, chlorodifluoroacetic acid, 2,2,3,3-tetrafluoropropionic acid, 3,3,3-trifluoropropionic acid, alpha,alpha,alpha-trifluorotoluic acid, pentafluorobenzoic acid, and anhydrides thereof.

The amount of polyester polymer utilized is not limited. However, one advantage of the method of this invention is that sensitivity and accuracy of the method is such that only a small amount of polyester polymer is required to obtain quantitative measurement of vinyl ends concentration. Typically, the amount of polyester polymer combined with liquid solution ranges from at least 0.01 grams, or at least 0.05 grams, or at least 0.1 grams, or at least 0.3 grams, and up to about 1.0 grams, or up to about 5.0 grams, or up to about 10 grams, or up to about 100 grams. The amount of liquid solution is generally an amount required to dissolve the polyester polymer at ambient temperatures. The amount of fluorinated or perfluorinated acid or anhydride is generally an amount necessary to provide a sufficient number of fluorinated or perfluorinated carboxylic acid moieties (derivates from the acid or anhydride) to react with the number of vinyl ends in the polyester.

Perfluorinated acids and anhydrides, especially trifluoroacetic acid and trifluoroacetic anhydride and mixtures thereof, are particularly effective in the method of this invention due to their ability to solubilize or dissolve a broad number of polyester polymers. In the specific example of PET (polyethylene terephthalate) polymer and even more specifically PET polymer pellets with a high degree of crystallinity normally used for CSD and water bottle applications, it is difficult to find appropriate solvents in which to dissolve these pellets.

In U.S. Pat. No. 5,852,164, there is described a method for counting vinyl ends of a highly modified polyester polymer in a chloroform solvent using a 1H-NMR (proton nuclear magnetic resonance) which is similar or possibly the same as that described in reference A. Highly modified polyester polymers, for example polyethylene terephthalate modified with greater than 30 mole percent cyclohexanedimethanol are generally amorphous as opposed to crystalline. In the instance where in the measurement of vinyl ends for PET polymer pellets with a high degree of crystallinity and large average molecular weight, the methods described in U.S. Pat. No. 5,852,164 and reference A are generally not applicable since the polyester would either not dissolve in the chloroform solvent, or there would be a need to utilize excessive solvent to polyester weight ratios such that the sensitivity and/or accuracy of subsequent counting of vinyl ends would be impacted. Further the other methods described do not produce measurements of vinyl ends concentrations with the sensitivity or the specificity of the present invention. In particular, in discussing the utilized methods therein, Reference A indicates that a shortcoming of the coulometric bromination method is a lack of specificity toward vinyl ends. Further, in the discussion section of the Reference A, it is further speculated that the difference between concentration values obtained via the 1H-NMR method and the modified coulometric bromination method may be a result of either experimental error or a result of the presence of other unsaturated species in the polymer. Additionally, Reference A indicates that the shortcoming of the 1H-NMR method is a lack of sensitivity, as the vinyl end group could only be detected in PET samples subjected to significant thermal degradation. Presumably, thermal degradation of the polymer was necessary to increase the number of vinyl ends so that the 1H-NMR method could be used to obtain a quantitative measurement of vinyl ends.

In this invention, perfluorinated acids and anhydrides, especially trifluoroacetic acid and trifluoroacetic anhydride and mixtures thereof, are particularly useful since they create a chemical structure within the polymer that is sensitive to fluorine nuclear magnetic resonance (FMR) measurement and which produce a distinct FMR signal. Hence, unlike other methods, the method of this invention is both specific and sensitive to the counting of vinyl ends in polyester polymers. In particular, the sensitivity of the method is such that vinyl ends are detectable in a range from at least 0.01 millimoles per kilogram of polymer (mmol/kg), or at least 0.05 mmol/kg, or at least 0.1 mmol/kg, and up to about 0.5 mmol/kg, or up to about 10.0 mmol/kg, or up to about 100 mmol/kg, based on the mass of a polyester sample of about 0.5 grams. Compared with other methods, this invention is more sensitive and specific for measurement of the vinyl end group. The reported range of the measurement of the bromination method is 1 to 20 mmol/kg. The reported precision of the bromination method is 0.25 mmol/kg for vinyl ends concentration between 1 to 20 mmol/kg. The 1H-NMR method does not report a precision value or range of measurement.

Because of the high solubility of polyester polymers in perfluorinated acids and anhydrides, dissolving of the polyester polymer into the liquid solution can proceed unaided. However, in an embodiment of this invention, it is provided that the combining of polyester polymer with liquid solution can be subjected to agitation and/or subjected to heating to hasten dissolution of the polyester polymer and reaction of the vinyl end groups. Agitation can be accomplished by any means known in the art. For example, agitation can be accomplished by mechanical stirring. Further, heating can be accomplished by any means known in the art. In a preferred embodiment, the liquid solution or the mixture of liquid solution and polymer may be heated up to about 30° C., or up to about 50° C., or up to about 150° C., or up to about 300° C. In the instance where the liquid solution comprises trifluoroacetic acid and trifluoroacetic anhydride under anhydrous conditions, heating and agitation may be used to hasten reaction of the vinyl end groups with excess perfluorinated moieties.

The liquid solution and/or mixture of liquid solution with polymer may also comprise other chemical compounds. For example, the liquid solution may be a mixture of trifluoroacetic acid, trifluoroacetic anhydride, and a solvent. Particularly suitable solvents include liquids in which polyester polymers exhibit some solubility (chloroform, for example). In general, suitable solvents may include aliphatic and aromatic hydrocarbons, esters, and ethers. These solvents may be halogenated derivatives of aliphatic hydrocarbons, esters, or ethers. Further, any hydrogen atoms that comprise the molecules of these solvents may be substituted with or replaced with deuterium or tritium. The liquid solution and/or mixture of liquid solution with polymer may also comprise other chemical compounds comprising fluorine, in as far as the presence of those fluorine-containing compounds do not interfere with the measurement of vinyl ends concentration. Examples of suitable compounds include, but are not limited to, butane, pentane, hexane, heptane, octane, nonane, decane, undecane, dodecane, cyclopentane, cyclohexane, cycloheptane, 2,2,4-trimethyl pentane, acetonitrile, butyl acetate, tert-butyl methyl ether, 1,4-dioxane, ethyl ether, isopropyl ether, butyl ether, 2-methoxyethyl ether, tetrahydrofuran, nitromethane, benzene, toluene, xylene, ethylbenzene, nitrobenzene, benzonitrile, anisole, pyridine, chlorobenzene, bromobenzene, dichlorobenzene, trichlorobenzene, dibromobenzene, methylene chloride, methylene chloride-d2, chloroform, chloroform-d, chlorobutane, carbon tetrachloride dichloromethane, dichloroethane, dichloroethylene, trichloroethane, trichloroethylene, tetrachloroethane, tetrachloroethane-d2, tetrachloroethylene chlorodifluoroacetic acid.

Additionally, the presence of water or alcohols in the liquid solution and/or mixture of liquid solution with polymer is discouraged. Fluorinated and perfluorinated acid moieties react with vinyl ends on the polymer. The presence of water or alcohols in the liquid may lead to undesirable chemical reactions. For example, the presence of water may lead to the formation of hydrolysis products. Under some circumstances, for example when using a liquid solution and/or mixture of liquid solution with polymer comprising a perfluorinated carboxylic acid anhydride such as trifluoroacetic anhydride, it may be preferable to utilize a stoichiometric excess of anhydride. However, a large excess of anhydride is generally not desirable. For example, when a liquid solution comprising trifluoroacetic acid and trifluoroacetic anhydride is utilized, subsequent analysis of quantitative FMR signals for specific fluorine compounds indicative of vinyl ends may be indistinguishable FMR signals associated with trifluoracetic anhydride.

In the instance where an excess amount of fluorinated or prefluorinated acid moieties are utilized, there is provided embodiments of the present invention wherein the excess amount of fluorinated or perfluorinated acid moieties are either removed or reacted to a compound or compounds that do not interfere in the quantitative measurement of vinyl ends. For example, when a liquid solution and/or mixture of liquid solution with polymer comprises trifluoroacetic acid and trifluoroacetic anhydride, it may be advantageous to reduce the concentration of trifluoroacetic anhydride moieties prior to subjecting the mixture to an analysis wherein a quantitative signal for fluorine is produced. If the mixture is subjected to FMR (fluorine nuclear magnetic resonance) measurement, the trifluoroacetic anhydride can cause problems with measurements due to its large signal size in the FMR spectrum compared with the vinyl ends group signal. However, it also may be advantageous for a small amount of trifluoroacetic anhydride to remain in the mixture. A slight excess of trifluoroacetic anhydride can be used to prevent hydrolysis from introduction of water (i.e. maintain anhydrous conditions) and to ensure that the reaction of trifluoroacetic acid moieties with vinyl ends is complete. In a preferred embodiment, the molar ratio of fluorinated or perfluorinated acid moieties to vinyl end moieties ranges from about at least 1:1 (acid moieties:vinyl end moieties), or about at least 5:1, or about at least 10:1, and up to about 1000:1, or up to about 10000:1, or up to about 30000:1.

The method by which excess fluorinated or perfluorinated moieties is removed can be any method known in the art. For example, excess fluorinated or perfluorinated moieties (e.g. fluorinated or perfluorinated carboxylic acid or anhydries) can be removed by evaporation or distillation. For example, when the liquid solution and/or mixture of liquid solution with polymer comprises trifluoroacetic anhydride and trifluoroacetic acid, it is possible to remove the anhydride by heating the solution thereby evaporating the anhydride preferentially since it has a substantially lower boiling point than the acid. In another example, it may be possible to remove the anhydride using a distillation column with a condenser and reflux head thereby selectively fractionating low boiling components such as trifluoroacetic anhydride from higher boiling components. Additionally, it is provided that following removal of any fluorinated or perfluorinated moieties, an amount of any compound comprising said fluorinated or perfluorinated moieties can be added back into the liquid solution or any mixture derived therefrom either directly or indirectly to form a mixture with a small percentage excess of fluorinated or perfluorinated moieties.

In a preferred embodiment of the present invention, excess amount of fluorinated or prefluorinated acid moieties can be reacted to a compound or compounds that do not interfere in the quantitative measurement of vinyl ends. For example, when using a liquid solution and/or mixture of liquid solution with polymer comprising trifluoroacetic acid and anhydride, a non-fluorinated organic acid may be added that can react with any excess anhydride. Suitable organic acids include, but are not limited to, unsubstituted or substituted, saturated, aliphatic or aromatic carboxylic acids containing a total of up to about 20 carbon atoms. The unsubstituted organic acids typically contain 2 to 18, preferably about 2 to 6, carbon atoms. The organic acid may be substituted with one or more, typically not more than one, substituent selected from alkoxy containing up to about 12 carbon atoms, halogen such as chlorine and bromine, containing up to about 12 carbon atoms. The organic acid may be substituted with a second carboxyl group, e.g., adipic acid, azelaic acid and the like. The organic acid preferably is an unsubstituted alkanoic acid containing about 2 to 6 carbon atoms (e.g. acetic acid, propanoic acid, n-butanoic acid, etc. . . . ). The organic acid may also include mixtures of suitable organic acids. For example, a mixture containing two or more alkanoic acids containing about 2 to 6 carbon atoms (e.g. a mixture containing approximately 90% acetic acid and 10% propanoic acid) may be used. The organic acid may be used amounts such that the non-fluorinated acid moieties to fluorinated moiety ratios are in the range of from about at least 2 parts non-fluorinated acid moieties to 1 part fluorinated acid moieties, or from about 3 parts non-fluorinated acid moieties to 1 part fluorinated acid moieties and up to about 8 parts non-fluorinated acid moieties to 1 part fluorinated acid moieties, or up to about 10 parts non-fluorinated acid moieties to 1 part fluorinated acid moieties

It is also provided in another embodiment of this invention, the addition or presence of at least one chemical compound that can function as a standard by which quantitative signals for fluorine or fluorine compounds are compared with. For example, in the specific instance where FMR is utilized, quantitative signals for specific fluorine compounds are often compared against quantitative signals for another chemical compound containing fluorine that is present in known amounts. The number of fluorine compounds is not limited. However, suitable fluorine compounds include any fluorinated and/or perfluorinated compound which is a liquid at ambient conditions and exhibit none or minimal chemical reactivity with other compounds in the liquid mixture. Preferably, the fluorinated and/or perfluorinated compound or compounds exhibit a signal within a FMR spectra that is distinct (i.e. does not overlap) with respect to other fluorine-containing compounds in the liquid mixture. Preferred are compounds containing fluorine such as, but not limited to, alpha,alpha,alpha-trifluorotoluene, alpha,alpha,alpha-trifluorotoluic acid, (trifluoromethoxy)benzene, (trifluoromethoxy)toluene, alpha,alpha,alpha-trifluorotolunitrile, hexafluorobenzene, octafluorotoluene, trifluoroacetonitrile, trifluoroacetamide.

Once a suitable mixture comprising the polyester polymer and at least one fluorinated carboxylic acid or fluorinated carboxylic anhydride is produced, the mixture is subjected to an analysis wherein a quantitative signal for fluorine or fluorine compounds which have reacted with the vinyl end group is produced. There are many known techniques by which a signal for fluorine is produced. Because fluorine has a nuclear spin of one-half, a strong signal, and a large coupling constant, NMR (nuclear magnetic resonance) spectroscopic analysis is a preferred. Even more preferable is FMR. Other means for providing a specific signal for fluorine analyte reacted with a vinyl end group may include, but are not limited to, gas chromatography, infrared spectroscopy, Raman spectroscopy, and mass spectrometry. In general, a commercially available instrument (e.g. a FMR instrument, an NMR instrument, a mass spectrometer, etc. . . . ) can be used to provide a quantitative signal.

Once a suitable signal is obtained, the quantity or concentration of vinyl ends in the polyester polymer is obtained by calculation. Typically, commercial instruments produce signal in the form of discrete counts or spectra. For example, an FMR instrument may produce quantitative data in the form of a spectra wherein the signal for the vinyl ends can be calculated from the area under a curve. The area signal can measured with appropriate known techniques and compared with the area of signal for known standards as part of calculation to measure the quantity and/or concentration of vinyl ends in a polyester polymer.

Once suitable calculations are completed, the numerical value or values for the concentration or amount of vinyl ends in the polyester polymer is reported or displayed. The numerical value may be reported or recorded onto a display such as a computer screen or monitor. The numerical value or a range of numerical values may be reported or recorded onto archiving media. Archiving media would be any tangible object used to record the numerical value for access at a later time. Examples of archiving media include, but are not limited to, paper notebooks, paper journals, computer magnetic storage drives, computer optical storage drives, and memory chips.

This invention can be further illustrated by the additional examples of embodiments thereof, although it will be understood that these examples are included merely for purposes of illustration and are not intended to limit the scope of the invention.

### EXAMPLES

### Example 1

The measurement of the vinyl end group in a poly(ethyleneterephthalate) polyester is completed as follows.

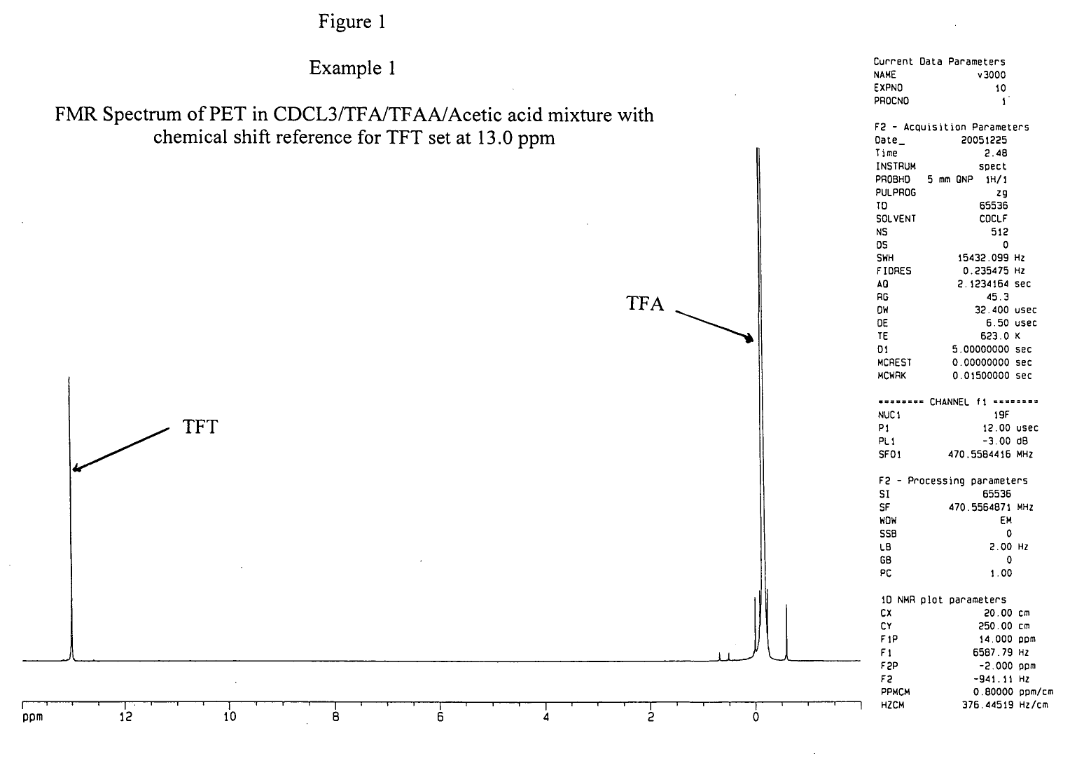

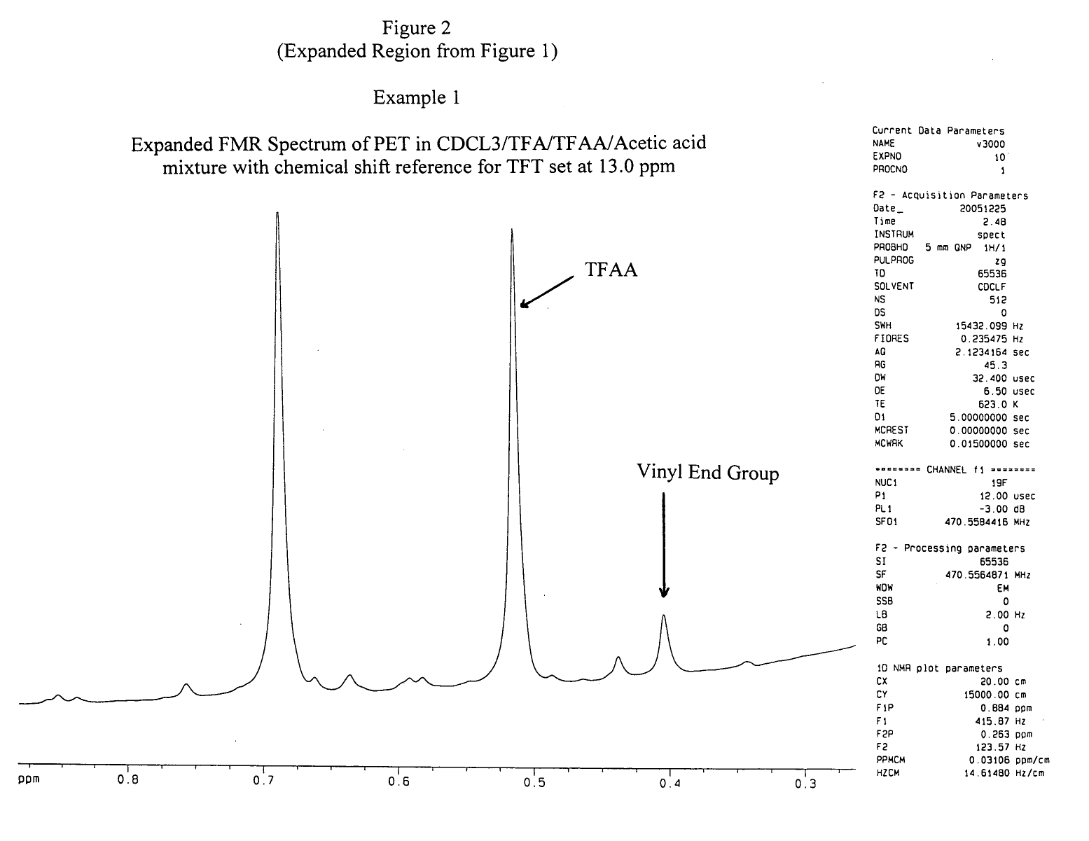

Approximately 0.4 grams of the sample is weighed to the nearest mg and placed in a 4 dram screw top vial along with a Teflon coated stir bar. A fresh solvent mixture (solution A) is prepared with exact volume ratios by measuring 75 parts Chloroform-d (CDCL3, Aldrich Chemical Company), 19 parts Trifluoroacetic acid (TFA), and 6 parts Trifluoroacetic anhydride (TFAA). Exactly 4.00 ml of the solution A is added to the sample vial and the vial is closed and sealed with a polycone screw top closure. The vial is heated to 50° C. in an aluminum heat block and stirred for 16 hrs. The vial is then removed from the heat block and cooled. A fresh solution (solution B) is prepared with exact volume ratios by mixing 2 parts of solution A and one part acetic acid. The vial closure is opened and exactly 1.00 ml of solution B and exactly 50 microliters of alpha,alpha,alpha-trifluorotoluene (TFT) is added to the vial. The vial is closed and mixed well. A portion of the prepared solution is loaded into a NMR tube, and a NMR spectrum is recorded for analysis on a Bruker Avance 500 MHz instrument using conditions which provide quantitative signals for the fluorine 19 NMR experiment or a NMR instrument with similar capability. Key NMR instrument conditions are; Pulse delay=5 sec.; Sweep width=32.795 ppm; Number of scans averaged=512; Number of points=65536; Line broading=2.0 Hz. Representative spectra are shown in FIGS. 1 and 2. The chemical shift is referenced at 13.0 ppm with the alpha,alpha,alpha-trifluorotoluene peak. The area of the alpha,alpha,alpha-trifluorotoluene peak at 13 ppm including the area of the spinning side bands is measured accurately along with the accurate area of the vinyl end group peak at approximately 0.4 ppm. If the vinyl end group signal is not baseline resolved from adjacent peaks, the area may be measured using curving fitting methods or other acceptable area measurement methods. In the formula below, a correction factor multiplier of 1.1 is used to correct the area of the vinyl end group to improve accuracy. The method standard deviation is 0.43 mmol/kg at the 5.6 mmol/kg vinyl end group level and 0.11 mmol/kg at the 0.76 mmol/kg vinyl end group level.

Representative calculations are shown below;

mmol/kg vinyl end group=((area of vinyl end group)*1.1*0.0595*1,000,000) divided by ((area of the TFT peak)*146.1*(sample weight in gm))

### Comparative Example 1

0.06 grams of crystallized PET pellets were added to 5 ml of chloroform and stirred and heated at 50° C. for 48 hrs. The pellets did not dissolve or change shape by observation.

What is claimed is:

1. A method for the measurement of vinyl ends concentration in a polyester polymer comprising:
a). dissolving said polyester polymer with a liquid solution comprising at least one fluorinated carboxylic acid or fluorinated carboxylic anhydride to form an anhydrous mixture;
b). optionally heating said liquid solution or said mixture to a temperature between about 30° C. and 300° C.;
c). subjecting said unheated or heated mixture to an analysis wherein a quantitative signal for fluorine or a fluorine-containing compound is produced; and
d). calculating the quantity or concentration of vinyl ends from said quantitative signals.

2. The method of claim 1, wherein said polyester polymer comprises a dicarboxylic acid moiety comprising terephthalic acid or its ester forming derivative and a diol moiety comprising ethylene glycol.

3. The method of claim 1, wherein said polyester polymer comprises a dicarboxylic acid comprising isophthalic acid or its ester forming derivative and a diol moiety comprising ethylene glycol.

4. The method of claim 1, wherein said polyester polymer comprises a dicarboxylic acid comprising naphthalene dicarboxylic acid or its ester forming derivative and a diol moiety comprising ethylene glycol.

5. The method of claim 1, wherein said fluorinated carboxylic acid comprises at least one of linear or branched aliphatic or aromatic carboxylic acids, wherein at least one of the carbon-bonded hydrogen atoms has been replaced by fluorine. As it is used herein, the term perfluorinated means that all of the carbon-bonded hydrogen atoms have been replaced by fluorine. Examples of suitable fluorinated acids include, but are not limited to, monofluoroacetic acid, difluoroacetic acid, trifluoroacetic acid, pentafluoropropionic acid, heptafluorobutyric acid, perfluoropentanoic acid, perfluorohexanoic acid, chlorodifluoroacetic acid, 2,2,3,3-tetrafluoropropionic acid, 3,3,3-trifluoropropionic acid, alpha,alpha,alpha-trifluorotoluic acid, pentafluorobenzoic acid, or mixtures thereof.

6. The method of claim 1, wherein said fluorinated carboxylic acid anhydride comprises at least one of linear or branched aliphatic or aromatic carboxylic acid anhydride or mixed anhydride, wherein at least one of the carbon-bonded hydrogen atoms has been replaced by fluorine. As it is used herein, the term perfluorinated means that all of the carbon-bonded hydrogen atoms have been replaced by fluorine. Examples of suitable fluorinated anhydrides include, but are not limited to, monofluoroacetic anhydride, difluoroacetic anhydride, trifluoroacetic anhydride, pentafluoropropionic anhydride, heptafluorobutyric anhydride, perfluoropentanoic anhydride, perfluorohexanoic anhydride, chlorodifluoroacetic anhydride, 2,2,3,3-tetrafluoropropionic anhydride, 3,3,3-trifluoropropionic anhydride, alpha,alpha,alpha-trifluorotoluic anhydride, pentafluorobenzoic anhydride, or mixtures thereof.

7. The method of claim 1, wherein the molar ratio or fluorinated carboxylic acid moieties to vinyl end groups in said liquid solution or said mixture is in the amount between 1:1 and 30000:1.

8. The method of claim 7, wherein the molar ratio or fluorinated carboxylic acid moieties to vinyl end groups in said liquid solution or said mixture is in the amount between 5:1 and 10000:1.

9. The method of claim 8, wherein the molar ratio or fluorinated carboxylic acid moieties to vinyl end groups in said liquid solution or said mixture is in the amount between 10:1 and 1000:1.

10. The method of claim 1, wherein said liquid solution or said mixture comprises at least one solvent wherein said solvent comprises at least one of butane, pentane, hexane, heptane, octane, nonane, decane, undecane, dodecane, cyclopentane, cyclohexane, cycloheptane, 2,2,4-trimethyl pentane, acetonitrile, butyl acetate, tert-butyl methyl ether, 1,4-dioxane, ethyl ether, isopropyl ether, butyl ether, 2-methoxyethyl ether, tetrahydrofuran, nitromethane, benzene, toluene, xylene, ethylbenzene, nitrobenzene, benzonitrile, anisole, pyridine, chlorobenzene, bromobenzene, dichlorobenzene, trichlorobenzene, dibromobenzene, methylene chloride, methylene chloride-d2, chloroform, chloroform-d, chlorobutane, carbon tetrachloride dichloromethane, dichloroethane, dichloroethylene, trichloroethane, trichloroethylene, tetrachloroethane, tetrachloroethane-d2, tetrachloroethylene, or chlorodifluoroacetic acid.

11. The method of claim 1, wherein said liquid solution or said mixture comprises at least one chemical compound standard in known quantity wherein said standard comprises at least one of alpha,alpha,alpha-trifluorotoluene, alpha,alpha,alpha-trifluorotoluic acid, (trifluoromethoxy)benzene, (trifluoromethoxy)toluene, alpha,alpha,alpha-trifluorotolunitrile, hexafluorobenzene, octafluorotoluene, trifluoroacetonitrile, trifluoroacetamide, or mixtures thereof.

12. The method of claim 1, wherein said quantitative signal is produced by one or more nuclear magnetic resonance instruments.

13. The method of claim 12, wherein said nuclear magnetic resonance instrument comprises a fluorine nuclear magnetic resonance instrument.

14. The method of claim 1, wherein said quantitative signal is produced by one or more gas chromatography instruments.

15. The method of claim 1, wherein quantitative signal is produced by one or more infrared spectroscopy instruments.

16. The method of claim 15, wherein said infrared spectroscopy instrument comprises a fourier transform infrared spectrometer.

17. The method of claim 1, wherein quantitative signal is produced by one or more Raman spectroscopy instruments.

18. The method of claim 1, wherein the dissolving of polyester polymer is in the amount of between 0.01 grams and 100 grams of polyester polymer.

19. The method of claim 18, wherein the dissolving of polyester polymer is in the amount of between 0.05 grams and 10 grams of polyester polymer.

20. The method of claim 19, wherein the dissolving of polyester polymer is in the amount of between 0.1 grams and 5 grams of polyester polymer.

21. The method of claim 1 comprising the additional step of removing excess fluorinated moieties from said liquid solution or from said mixture.

22. The method of claim 21, wherein said removing of excess fluorinated moieties from said liquid solution or from said mixture is by evaporation.

23. The method of claim 21, wherein said removing of excess fluorinated moieties from said liquid solution or from said mixture is by distillation.

24. The method of claim 1, comprising the additional step of reacting excess fluorinated moieties within said liquid solution or within said mixture with a non-fluorinated organic acid.

25. The method of claim 24, wherein said non-fluorinated organic acid comprises at least one of unsubstituted or substituted, saturated, aliphatic or aromatic carboxylic acids containing a total of up to about 20 carbon atoms. The unsubstituted organic acids typically contain 2 to 18, preferably about 2 to 6, carbon atoms. The organic acid may be substituted with one or more, typically not more than one, substituent selected from alkoxy containing up to about 12 carbon atoms, halogen such as chlorine and bromine, containing up to about 12 carbon atoms. Examples of suitable compounds include, but are not limited to acetic acid, propionic acid, butyric acid, valeric acid, benzoic acid, toluic acid, or mixtures thereof.

26. The method of claim 1, wherein said heating of said liquid solution or said mixture is to a temperature between about 50° C. and 150° C.

27. A method of describing a vinyl ends concentration or amount within a polyester polymer as a numerical value comprising reporting said numerical value in or on an archiving media or display, said numerical value having been obtained by the method of claim 1.

28. A method of describing a vinyl ends concentration or amount within a polyester polymer as a numerical range comprising reporting said numerical range in or on an archiving media or display, said numerical range having been obtained by the method of claim 1.

29. The method of claim 27 or 28, wherein said archiving media comprises at least one of paper notebooks, paper journals, computer magnetic storage drives, computer optical storage drives, memory chips or a combination thereof.

30. The method of claim 27 or 28, wherein said display comprises a computer screen or computer monitor.
